(19) United States
(12) Patent Application Publication
(10) Pub. No.: US 2025/0079024 A1
(43) Pub. Date: Mar. 6, 2025

(54) METHOD FOR GENERATING INFECTIOUS DISEASE PREDICTION KEYWORD THAT CHANGES OVER TIME BASED ON WORD EMBEDDING AND APPARATUS PERFORMING THE SAME

(71) Applicant: THE CATHOLIC UNIVERSITY OF KOREA INDUSTRY-ACADEMIC COOPERATION FOUNDATION, Seoul (KR)

(72) Inventors: Dong Hwa JEONG, Bucheon-si (KR); Kang Min KIM, Siheung-si (KR); Seong Ho AHN, Bucheon-si (KR); Kwang Il YIM, Seoul (KR)

(73) Assignee: THE CATHOLIC UNIVERSITY OF KOREA INDUSTRY-ACADEMIC COOPERATION FOUNDATION, Seoul (KR)

(21) Appl. No.: 18/623,731

(22) Filed: Apr. 1, 2024

(30) Foreign Application Priority Data
Aug. 28, 2023 (KR) 10-2023-0112512

(51) Int. Cl. G16H 50/80; G06N 20/00

(52) U.S. Cl. CPC G16H 50/80; G06N 20/00

(57) ABSTRACT

The method comprises obtaining a document including a target infectious disease as a corpus for each of a plurality of time sections, converting a plurality of words included in the obtained corpus into embedding vectors, calculating similarities between the respective converted embedding vectors and an embedding vector indicating the target infectious disease, extracting an embedding vector of which the calculated similarity is higher than a predetermined first threshold value, for each time section, obtaining first time series data indicating a search volume, over time, of a word corresponding to the embedding vector extracted for each time section, calculating a correlation coefficient between the obtained first time series data and second time series data, and generating a word corresponding to the first time series data of which the calculated correlation coefficient is higher than a predetermined second threshold value as a keyword for each time section.

### CROSS-REFERENCE TO RELATED APPLICATION

This application claims priority from Korean Patent Application No. 10-2023-0112512 filed on Aug. 28, 2023 in the Korean Intellectual Property Office, and all the benefits accruing therefrom under 35 U.S.C. 119, the contents of which in its entirety are herein incorporated by reference.

### BACKGROUND

### 1. Technical Field

The present disclosure relates to a method for generating an infectious disease prediction keyword that changes over time based on word embedding and an apparatus for performing the same, and more particularly, to a method for automatically generating a keyword to be input to an infectious disease prediction model using online search volume data of words with a high similarity to an infectious disease and data on the number of confirmed cases of the infectious disease in an embedding space.

### 2. Description of the Related Art

When an infectious disease spreads, most countries utilize the number of daily confirmed cases of the infectious disease as an important index in determining a quarantine policy. For example, since coronavirus disease-19 (COVID-19) began to spread globally in 2020, the quarantine policy has had a significant influence on people's lives and economic activities such as an increase in delivery food order and activation of telemedicine and telecommuting. In other words, when the infectious disease is prevalent, the number of daily confirmed cases is an important measure of national policies and people's lives. Accordingly, it is very important to predict the number of confirmed cases of the infectious disease.

Recently, it has been proven that online human behavioral aspects, particularly, an online search and communication on a social network service (SNS), have a statistically significant association with the spread of the infectious disease. In particular, a search word ranking on a portal site is one of the important data used to predict the infectious disease. However, in most studies, a keyword for predicting the infectious disease has been selected based on empirical human knowledge. Therefore, a similarity between the selected keyword and the infectious disease, which is a prediction target, might not be objectively quantified, and temporal variability of search words could not be taken into account.

### SUMMARY

Aspects of the present disclosure provide a method for automatically generating a keyword for predicting an infectious disease from an online document by reflecting person's interests that change over time.

Aspects of the present disclosure also provide a method for training an infectious disease prediction model that may adapt to a temporal change through automated keyword generation without expert experience.

According to some embodiments of the present disclosure, there is provided a method for generating an infectious disease prediction keyword, the method being performed by a computing apparatus. The method comprises obtaining a document including a target infectious disease as a corpus for each of a plurality of time sections, converting a plurality of words included in the obtained corpus into embedding vectors, calculating similarities between the respective converted embedding vectors and an embedding vector indicating the target infectious disease, extracting an embedding vector of which the calculated similarity is higher than a predetermined first threshold value, for each time section, obtaining first time series data indicating a search volume, over time, of a word corresponding to the embedding vector extracted for each time section, calculating a correlation coefficient between the obtained first time series data and second time series data indicating the number of confirmed cases of the target infectious disease over time, and generating a word corresponding to the first time series data of which the calculated correlation coefficient is higher than a predetermined second threshold value as a keyword for each time section.

In some embodiments, the converting of the plurality of words included in the obtained corpus into the embedding vectors and the calculating of the similarities are performed by a Word2Vec algorithm.

In some embodiments, the calculating of the correlation coefficient includes interpolating missing values of the first time series data and the second time series data, normalizing the first time series data and the second time series data, calculating correlation coefficients between the first time series data and the second time series data for each of a plurality of sliding windows, and determining a maximum value of the calculated correlation coefficients as the correlation coefficient between the first time series data and the second time series data.

In some embodiments, the normalizing of the first time series data and the second time series data is performed by a min-max algorithm.

In some embodiments, the generating of the word corresponding to the first time series data of which the calculated correlation coefficient is higher than the predetermined second threshold value as the keyword for each time section includes calculating a p-value of the calculated correlation coefficient when the calculated correlation coefficient is higher than the second threshold value, and generating a word corresponding to the first time series data of which the calculated p-value is lower than a predetermined third threshold value as the keyword for each time section.

In some embodiments, the generating of the word corresponding to the first time series data of which the calculated correlation coefficient is higher than the predetermined second threshold value as the keyword for each time section further includes determining a point in time when the correlation coefficient calculated within each time section is highest for the generated keyword.

In some embodiments, the method further comprises training an infectious disease prediction model using the generated keyword.

In some embodiments, the infectious disease prediction model is implemented as a regression model, and the training of the infectious disease prediction model includes regularizing a regularizer of the infectious disease prediction model.

In some embodiments, the method further comprises displaying the number of confirmed cases of the target infectious disease on a user terminal, the number of confirmed cases of the target infectious disease being predicted by inputting a keyword generated for a period input by a user.

According to another embodiments of the present disclosure, there is provided a computing apparatus. The apparatus comprises a processor, and a memory storing instructions, wherein when the instructions are executed by the processor, the instructions cause the processor to perform obtaining a document including a target infectious disease as a corpus for each of a plurality of time sections, converting a plurality of words included in the obtained corpus into embedding vectors, calculating similarities between the respective converted embedding vectors and an embedding vector indicating the target infectious disease, extracting an embedding vector of which the calculated similarity is higher than a predetermined first threshold value, for each time section, obtaining first time series data indicating a search volume, over time, of a word corresponding to the embedding vector extracted for each time section, calculating a correlation coefficient between the obtained first time series data and second time series data indicating the number of confirmed cases of the target infectious disease over time, and generating a word corresponding to the first time series data of which the calculated correlation coefficient is higher than a predetermined second threshold value as a keyword for each time section.

In some embodiments, calculating correlation coefficient includes interpolating missing values of the first time series data and the second time series data, normalizing the first time series data and the second time series data, calculating correlation coefficients between the first time series data and the second time series data for each of a plurality of sliding windows, and determining a maximum value of the calculated correlation coefficients as the correlation coefficient between the first time series data and the second time series data.

In some embodiments, generating the word corresponding to the first time series data of which the calculated correlation coefficient is higher than the predetermined second threshold value as the keyword for each time section includes calculating a p-value of the calculated correlation coefficient when the calculated correlation coefficient is higher than the second threshold value, and generating a word corresponding to the first time series data of which the calculated p-value is lower than a predetermined third threshold value as the keyword for each time section.

In some embodiments, when the instructions are executed by the processor, the instructions cause the processor to further perform training an infectious disease prediction model using the generated keyword, and displaying the number of confirmed cases of the target infectious disease on a user terminal, the number of confirmed cases of the target infectious disease being predicted by inputting a keyword generated for a period input by a user to the infectious disease prediction model.

In some embodiments, displaying the number of confirmed cases of the target infectious disease on the user terminal includes an operation of displaying the number of confirmed cases of the target infectious disease for a region input by the user on the user terminal.

In some embodiments, displaying the number of confirmed cases of the target infectious disease on the user terminal further includes an operation of displaying information related to the number of confirmed cases of the target infectious disease for a keyword of interest input by the user on the user terminal.

### BRIEF DESCRIPTION OF THE DRAWINGS

The above and other aspects and features of the present disclosure will become more apparent by describing in detail exemplary embodiments thereof with reference to the attached drawings, in which:

FIG. 1 is a block diagram illustrating an illustrative configuration of an apparatus for generating an infectious disease prediction keyword according to an exemplary embodiment of the present disclosure;

FIG. 2 is a flowchart illustratively illustrating a method for generating an infectious disease prediction keyword according to an exemplary embodiment of the present disclosure;

FIG. 3 illustrates an illustrative source code for performing converting a plurality of words into embedding vectors and calculating similarities in FIG. 2;

FIG. 4 illustrates an illustrative source code for performing obtaining first time series data in FIG. 2;

FIG. 5 is a flowchart specifically illustrating calculating a correlation coefficient in FIG. 2;

FIG. 6 illustrates an illustrative source code for performing normalizing first time series data and second time series data and calculating a correlation coefficient in FIG. 5;

FIG. 7 is a flowchart specifically illustrating generating a word corresponding to first time series data of which a calculated correlation coefficient is higher than a predetermined second threshold value as a keyword for each time section in FIG. 2;

FIG. 8 illustrates an illustrative user interface for performing displaying the number of confirmed cases of a target infectious disease on a user terminal in FIG. 2; and

FIG. 9 is a block diagram illustrating a hardware configuration of a computing apparatus for generating an infectious disease prediction keyword according to an exemplary embodiment of the present disclosure.

### DETAILED DESCRIPTION OF THE EMBODIMENTS

Hereinafter, preferred embodiments of the present disclosure will be described with reference to the attached drawings. Advantages and features of the present disclosure and methods of accomplishing the same may be understood more readily by reference to the following detailed description of preferred embodiments and the accompanying drawings. The present disclosure may, however, be embodied in many different forms and should not be construed as being limited to the embodiments set forth herein. Rather, these embodiments are provided so that this disclosure will be thorough and complete and will fully convey the concept of the disclosure to those skilled in the art, and the present disclosure will only be defined by the appended claims.

In adding reference numerals to the components of each drawing, it should be noted that the same reference numerals are assigned to the same components as much as possible even though they are shown in different drawings. In addition, in describing the present disclosure, when it is determined that the detailed description of the related well-known configuration or function may obscure the gist of the present disclosure, the detailed description thereof will be omitted.

Unless otherwise defined, all terms used in the present specification (including technical and scientific terms) may be used in a sense that can be commonly understood by those skilled in the art. In addition, the terms defined in the commonly used dictionaries are not ideally or excessively interpreted unless they are specifically defined clearly. The terminology used herein is for the purpose of describing particular embodiments only and is not intended to be limiting of the disclosure. In this specification, the singular also includes the plural unless specifically stated otherwise in the phrase.

In addition, in describing the component of this disclosure, terms, such as first, second, A, B, (a), (b), can be used. These terms are only for distinguishing the components from other components, and the nature or order of the components is not limited by the terms. If a component is described as being “connected,” “coupled” or “contacted” to another component, that component may be directly connected to or contacted with that other component, but it should be understood that another component also may be “connected,” “coupled” or “contacted” between each component.

The terms “comprise”, “include”, “have”, etc. when used in this specification, specify the presence of stated features, integers, steps, operations, elements, components, and/or combinations of them but do not preclude the presence or addition of one or more other features, integers, steps, operations, elements, components, and/or combinations thereof.

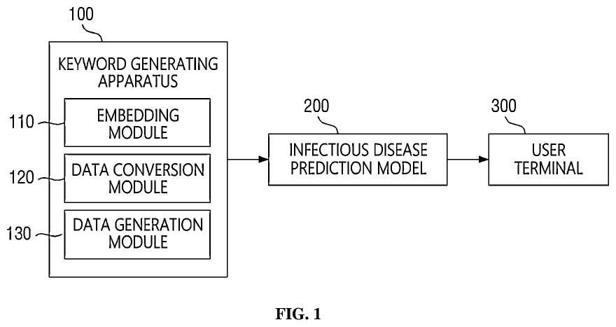

FIG. 1 is a block diagram illustrating an illustrative configuration of an apparatus 100 for generating an infectious disease prediction keyword according to an exemplary embodiment of the present disclosure. Hereinafter, the apparatus 100 for generating an infectious disease prediction keyword will be referred to as a keyword generating apparatus 100.

The keyword generating apparatus 100 may be a computing apparatus including one or more processors and memory. The keyword generating apparatus 100 may obtain an online document (e.g., an internet news, a social network service (SNS) post, etc.) including an infectious disease of which the number of daily confirmed cases is to be predicted (hereinafter referred to as a target infectious disease) as a corpus, convert words included in the obtained corpus into embedding vectors, extract words with a high similarity to a word indicating the target infectious disease, and then automatically generate a keyword required for predicting the infectious disease by converting the extracted words into online search volume data of a portal site over time and calculating a correlation coefficient between the online search volume data and the number of confirmed cases of the target infectious disease over time.

Referring to FIG. 1, the keyword generating apparatus 100 may include an embedding module 110, a data conversion module 120, and a data generation module 130. Meanwhile, it is to be noted that components (modules) of the keyword generating apparatus 100 illustrated in FIG. 1 indicate functional elements that are functionally divided and one or more components (modules) may be implemented in a form in which they are integrated with each other in an actual physical environment.

The embedding module 110 may obtain an online document including a word indicating the target infectious disease among online documents such as internet news or SNS posts as a corpus. In this case, the embedding module 110 may divide an entire period during which the online document including the target infectious disease is posted into a plurality of time sections and obtain a corpus for each of the plurality of time sections. For example, assuming that an internet news including “Corona” has been posted since January 2020, the embedding module 110 may divide a period from January 2020 to a survey reference date into a plurality of time sections and collect internet news including “Corona” for each time section. Here, a length of the time section may be determined so that a predetermined number or more of documents may be collected. For example, the internet news including “Corona” may be collected at monthly intervals and obtained as a corpus.

Thereafter, the embedding module 110 may convert words included in the obtained corpus into embedding vectors and calculate similarities between an embedding vector indicating the target infectious disease and other embedding vectors in an embedding space. For example, the embedding module 110 may convert the words included in the corpus into the embedding vectors using a Word2Vec algorithm. However, the present disclosure is not limited thereto, and the embedding module 110 may also perform word embedding-based similarity calculation on the words included in the corpus using another algorithm.

As a result of the similarity calculation, the embedding module 110 may extract an embedding vector of which the calculated similarity is higher than a predetermined first threshold value, for each time section. Here, the number of extracted embedding vectors may be determined depending on a size of the corpus, a method for implementing an infectious disease prediction model, and the like, and may affect accuracy of the infectious disease prediction model later. For example, the number of extracted embedding vectors may be adjusted through a parameter of a similarity calculation function of the Word2Vec algorithm. In addition, since the embedding module 110 obtains the corpus for each of the plurality of time sections as described above, the word indicated by the extracted embedding vector may change over time. The embedding module 110 may provide the embedded vector extracted for each time section to the data conversion module 120.

The data conversion module 120 may obtain first time series data indicating a search volume over time on the portal site for the word corresponding to the extracted embedding vector, using an application programming interface (API) provided by the portal site, and may provide the obtained first time series data the data generation module 130. The data generation module 130 may calculate a correlation coefficient between the obtained first time series data and second time series data indicating the actual number of confirmed cases of the target infectious disease over time. For example, the second time series data indicating the number of confirmed cases over time may be received from the national or local government. For example, the correlation coefficient may be a Pearson correlation coefficient, but the present disclosure is not limited thereto. The data generation module 130 may generate a word corresponding to the first time series data of which the calculated correlation coefficient is higher than a predetermined second threshold value as a keyword for each time section.

In order to calculate the correlation coefficient as described above, the data generation module 130 may perform preprocessing on the first time series data and the second time series data. For example, the data generation module 130 may interpolate missing values that may exist in the first time series data and the second time series data, and may normalize the first time series data and the second time series data through a min-max algorithm or the like. Thereafter, the data generation module 130 may calculate the correlation coefficient by applying a sliding window technique to the normalized first time series data and second time series data. For example, the data generation module 130 may determine a maximum value of correlation coefficients calculated for each of a plurality of sliding windows as a final correlation coefficient, and compare the final correlation coefficient with the predetermined second threshold value.

Next, the data generation module 130 may calculate a p-value for the correlation coefficient higher than the second threshold value for each time section, and may generate a word corresponding to the first time series data of which the calculated p-value is lower than a predetermined third threshold value as a keyword for each time section. For example, the third threshold value may be determined as one of the p-values that are a standard for statistical significance, such as 0.05, 0.01, 0.005, and 0.001.

Meanwhile, even though the keywords are extracted within the same time section, points in time when the correlation coefficient is highest among points in time of the plurality of time sections may be different from each other depending on the keywords. For example, a keyword indicating the infectious disease itself may have the highest correlation coefficient with the number of confirmed cases of the infectious at the same point in time as a point in time when the keyword is searched, but a keyword indicating a countermeasure against the infectious disease may have the highest correlation coefficient with the number of confirmed cases of the infectious disease may be highest a few days after or a few days before a point in time when the keyword is searched. Accordingly, not only the keyword for predicting the infectious disease may change over time, but a point in time when the infectious disease may be predicted may also change depending on the keyword.

Furthermore, through a process of calculating the correlation coefficient as described above, words that shows a high similarity to the target infectious disease in the embedding space but do not actually have relevance to the target infectious disease may be removed. For example, a postpositional particle in a Korean document or an article in an English document cannot but be calculated to have a high similarity to the target infectious disease in the embedding space, but may be removed as a word that does not have relevance to the target infectious disease in the process of calculating the correlation coefficient.

An infectious disease prediction model 200 may exist on a server positioned outside the keyword generating apparatus 100, may be trained by receiving the keyword for predicting the infectious disease generated from the keyword generating apparatus 100, and may predict and output the number of daily confirmed cases of the target infectious disease. For example, the infectious disease prediction model 200 may be implemented as a regression model (e.g., ElasticNet). However, the present disclosure is not limited thereto, and the infectious disease prediction model 200 may be implemented through an arbitrary deep learning algorithm as long as a sufficient number of keywords for training are secured. In particular, when the infectious disease prediction model 200 is implemented as the regression model, a regularizer of the regression model may be intensively regularized in a training process of the infectious disease prediction model 200. For example, as sparsity of the regularizer becomes higher, low-relevance data that could not be completely removed in a correlation coefficient calculation step may be removed.

Some of the keywords generated by the keyword generating apparatus 100 may be used for training of the infectious disease prediction model 200, and the others of the keywords generated by the keyword generating apparatus 100 may be used for verification of the infectious disease prediction model 200. For example, assuming that a length of each time section is 10 months, documents for the first 9 months may be used as training data, and documents for the next 1 month may be used as verification data. The present disclosure is not limited thereto, and a ratio between the training data and the verification data may change.

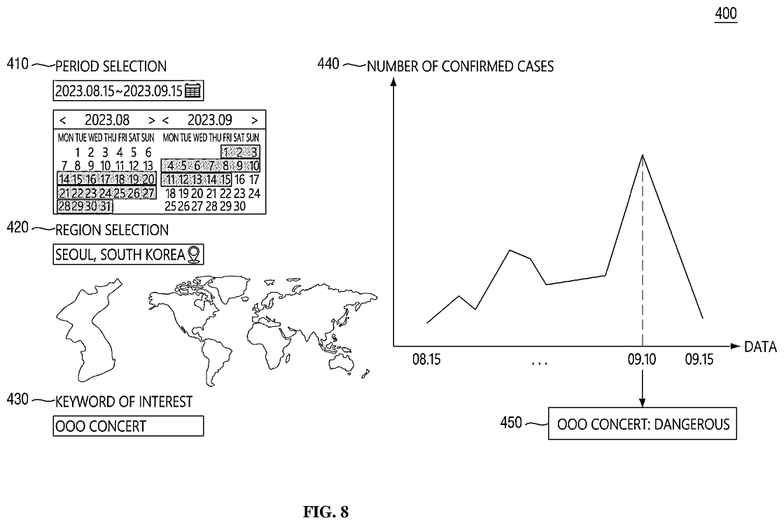

A user terminal 300 may be a computing apparatus used by a user in order to receive a prediction result for the number of confirmed cases of the target infectious disease from the infectious disease prediction model 200. For example, the user terminal 300 may include a smartphone, a desktop computer, a laptop computer, or the like. However, the present disclosure is not limited thereto, and the user terminal 300 may be implemented as any apparatus. For example, the user may input a period during which the number of confirmed cases of the target infectious disease is to be predicted, a region in which the numbers of confirmed cases of the target infectious disease is to be predicted, and a keyword that he/she is interested in through an application running on the user terminal 300, and the numbers of confirmed cases for each period and for each region may be displayed based on the user's input. An illustrative interface of such an application is described in detail with reference to FIG. 8.

In addition, components illustrated in FIG. 1 may communicate with each other through a network. For example, the network may be implemented as all types of wired/wireless networks such as a local area network (LAN), a wide area network (WAN), a mobile radio communication network, and a wireless broadband Internet (Wibro).

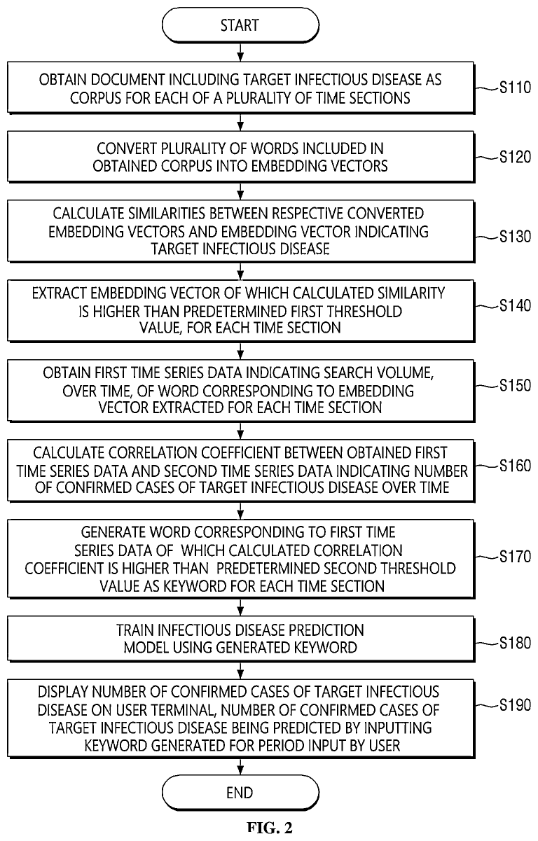

FIG. 2 is a flowchart illustratively illustrating a method for generating an infectious disease prediction keyword according to an exemplary embodiment of the present disclosure. For reference, FIG. 2 illustrates steps/operations performed in the keyword generating apparatus 100 of FIG. 1. Accordingly, in the following description, when a subject of a specific step/operation is omitted, it may be understood that the specific step/operation is performed by the keyword generating apparatus 100 of FIG. 1. Hereinafter, a description will be provided with reference to FIG. 1 along with FIG. 2.

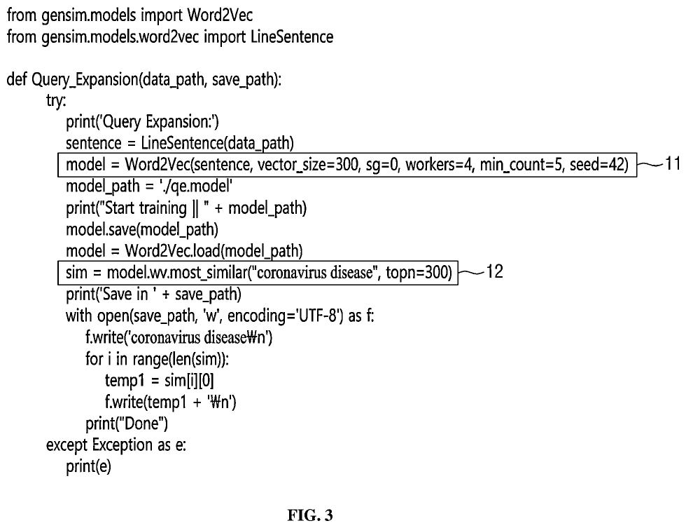

In S110, the embedding module 110 of the keyword generating apparatus 100 may obtain a document including a target infectious disease as a corpus for each of a plurality of time sections. As described above with reference to FIG. 1, the document including the target infectious disease may be an internet new or an SNS post. In S120, the embedding module 110 may convert a plurality of words included in the obtained corpus into embedding vectors. For example, the embedding module 110 may use the Word2Vec algorithm. In S130, the embedding module 110 may calculate similarities between the respective converted embedding vectors and an embedding vector indicating the target infectious disease. S120 and S130 will be described with reference to an illustrative source code in FIG. 3.

FIG. 3 illustrates an illustrative source code for performing converting a plurality of words into embedding vectors (S130) and calculating similarities (S130) in FIG. 2. Referring to FIG. 3, words of each sentence of the corpus may be converted into embedding vectors through the Word2Vec algorithm (11), topn (e.g., 300) similarities sim to the embedding vectors representing the target infectious disease (e.g., a coronavirus disease) may be calculated in descending order through a most_similar function of Word2Vec (12).

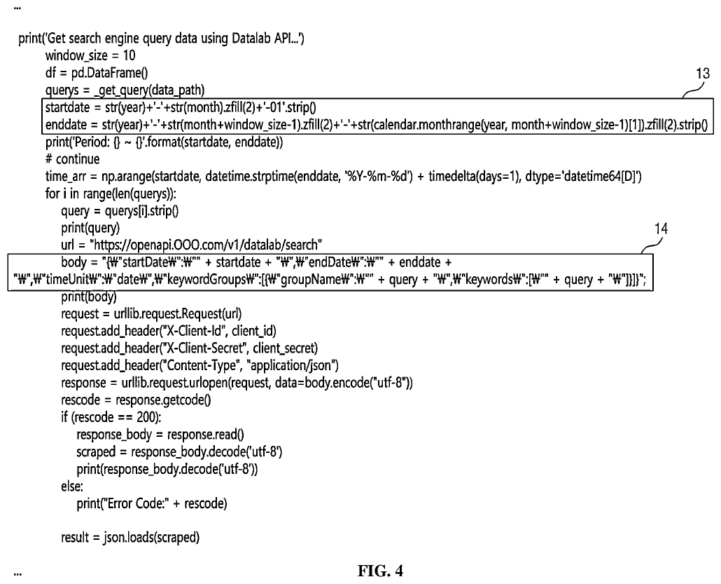

Referring to FIG. 2 again, in S140, the embedding module 110 may extract an embedding vector of which the calculated similarity is higher than a predetermined first threshold value, for each time section. Next, in S150, the data conversion module 120 of the keyword generating apparatus 100 may obtain first time series data indicating a search volume, over time, of a word corresponding to the embedding vector extracted for each time section. For example, the data conversion module 120 may obtain the first time series data using the API provided by the portal site. S150 will be described with reference to an illustrative source code in FIG. 4.

FIG. 4 illustrates an illustrative source code for performing obtaining first time series data (S150) in FIG. 2. Referring to FIG. 4, the words corresponding to the extracted embedding vector may be stored as queries, and a time section in which the words are to be converted to search volume data may be determined through a startdate and an enddate (13). Thereafter, search volume data of the word stored in the queries from the startdate to the enddate may be provided through openapi provided on the portal site (14). The search volume data provided as described above may correspond to the first time series data.

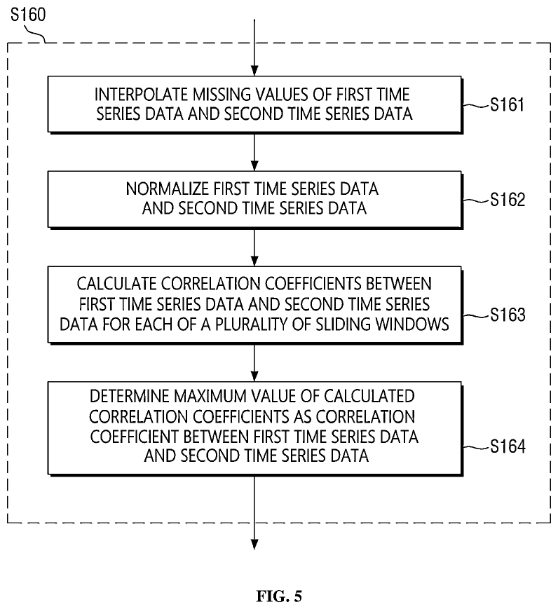

Referring to FIG. 2 again, in S160, the data generation module 130 may calculate a correlation coefficient between the obtained first time series data and second time series data indicating the number of confirmed cases of the target infectious disease over time. S160 will be described with reference to FIG. 5.

FIG. 5 is a flowchart specifically illustrating calculating a correlation coefficient (S160) in FIG. 2. In S161, the data generation module 130 may interpolate missing values of the first time series data and the second time series data, and in S162, the data generation module 130 may normalize the first time series data and the second time series data. For example, the data generation module 130 may normalize the first time series data and the second time series data using a min-max algorithm, but the present disclosure is not limited thereto.

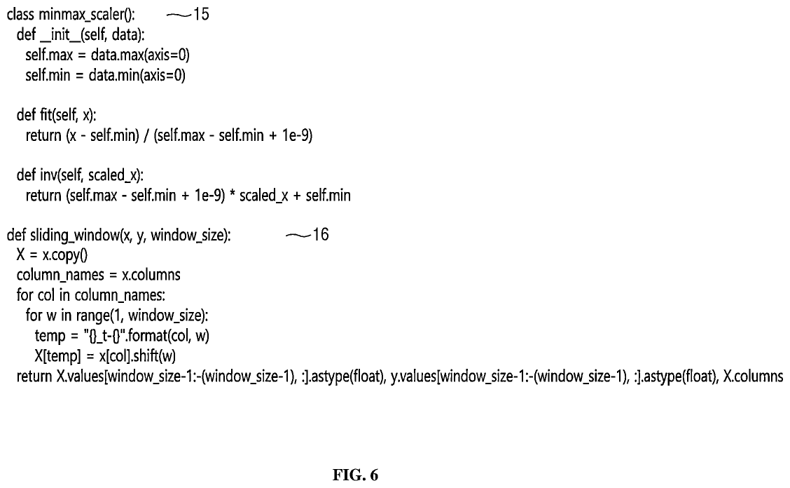

In S163, the data generation module 130 may calculate correlation coefficients between the first time series data and the second time series data for each of a plurality of sliding windows by applying a sliding window technique, and in step S164, the data generation module 130 may determine a maximum value of the correlation coefficients calculated for each sliding window as the correlation coefficient between the first time series data and the second time series data. Steps S162 to S163 will be described with reference to FIG. 6.

FIG. 6 illustrates an illustrative source code for performing normalizing first time series data and second time series data (S162) and calculating a correlation coefficient (S163) in FIG. 5. Referring to FIG. 6, a min-max function 15 for normalizing the first time series data and the second time series data and a function 16 for applying the sliding window technique in order to calculate the correlation coefficient are illustrated.

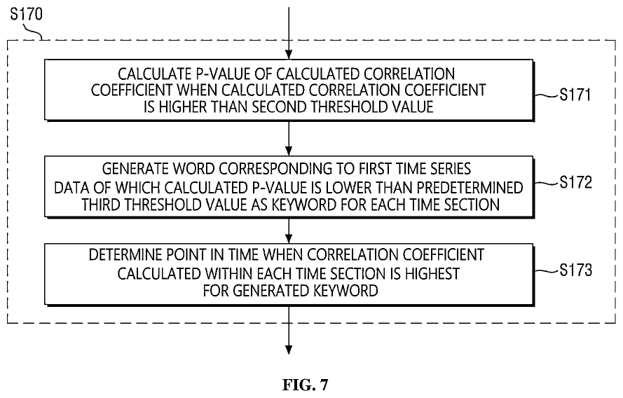

Referring to FIG. 2 again, in S170, the data generation module 130 may generate a word corresponding to the first time series data of which the calculated correlation coefficient is higher than a predetermined second threshold value as a keyword for each time section. S170 will be described with reference to FIG. 7.

FIG. 7 is a flowchart specifically illustrating generating a word corresponding to first time series data of which a calculated correlation coefficient is higher than a predetermined second threshold value as a keyword for each time section (S170) in FIG. 2. In S171, the data generation module 130 may calculate a p-value of the calculated correlation coefficient when the calculated correlation coefficient is higher than the second threshold value, and in S172, the data generation module 130 may generate a word corresponding to the first time series data of which the calculated p-value is lower than a predetermined third threshold value (0.05, 0.01, 0.005, 0.001, etc.) as the keyword for each time section. Then, in S173, the data generation module 130 may determine a point in time when the correlation coefficient calculated within each time section is highest (e.g., whether this point in time is a point in time when the keyword is searched or a point in time before or after the point in time when the keyword is searched) for the generated keyword.

Referring to FIG. 2 again, in S180, the infectious disease prediction model 200 may be trained using the generated keyword, and in S190, the number of confirmed cases of the target infectious disease predicted by inputting a keyword generated for a period input by the user to the infectious disease prediction model 200 may be displayed on the user terminal 300. S190 will be described with reference to FIG. 8.

FIG. 8 illustrates an illustrative user interface 400 for performing displaying the number of confirmed cases of a target infectious disease on a user terminal (S190) in FIG. 2. Referring to FIG. 8, the user may select a period for which the number of confirmed cases of the target infectious disease is to be predicted (410), may select a region (a domestic region or an overseas region) in which the number of confirmed cases of the target infectious disease is to be predicted (420), and may additionally input a keyword of interest (430). For example, when the keyword of interest input by the user is a concert and the date of the concert is within the period selected by the user, additional information such as information on whether it would be safe or dangerous for the user to go to the concert may be provided based on the number of confirmed cases of the target infectious disease. Alternatively, according to an exemplary embodiment, when the keyword of interest input by the user is a company related to the target infectious disease, additional information related to stocks of the company may be provided based on the number of confirmed cases of the target infectious disease.

For the period and the region input as described above, the number of daily confirmed cases predicted by the infectious disease prediction model 200 may be displayed (440), and the above-described additional information (e.g., information indicating that it is dangerous for the user to go to the concert because the expected number of confirmed cases is at its highest when the date of concert is September 10) may be output (450). The user interface 400 illustrated in FIG. 8 is an example, and the present disclosure is not limited thereto and the user interface 400 may also be implemented in a different form.

According to an exemplary embodiment of the present disclosure, it is possible to predict the number of confirmed cases of the target infectious disease by automatically generating the keyword related to the target infectious disease using the search volume data of the portal site over time without needing to directly select the keyword based on expert experience, and the same keyword is not generated for all time sections and different keywords are generated over time, and it is thus possible to reflect person's interests that change over time in the prediction of the target infectious disease.

Furthermore, according to an exemplary embodiment of the present disclosure, the region where the number of confirmed cases of the target infectious disease may be predicted is not limited to the domestic region, and an exemplary embodiment of the present disclosure may be applied to any region where online documents may be collected and statistics on the number of confirmed cases of the target infectious disease are provided. In addition, according to an exemplary embodiment of the present disclosure, the additional information on the keywords individually selected by the user in relation to the target infectious disease as well as the expected number of confirmed cases of the target infectious disease may be provided to the user.

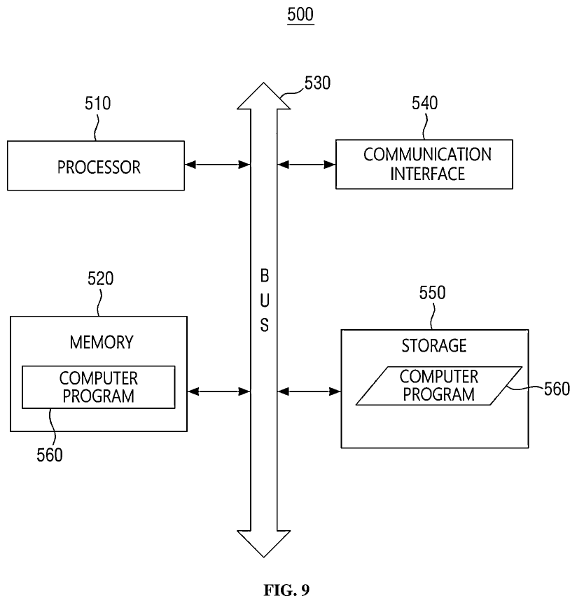

Until now, various embodiments of the present disclosure and effects according to the embodiments have been mentioned with reference to FIGS. 1 to 10. The effects of the technical idea of the present disclosure are not restricted to those set forth herein, and other unmentioned technical effects will be clearly understood by one of ordinary skill in the art to which the present disclosure pertains by referencing the detailed description of the present disclosure given below.

Although operations are shown in a specific order in the drawings, it should not be understood that desired results can be obtained when the operations must be performed in the specific order or sequential order or when all of the operations must be performed. In certain situations, multitasking and parallel processing may be advantageous. According to the above-described embodiments, it should not be understood that the separation of various configurations is necessarily required, and it should be understood that the described program components and systems may generally be integrated together into a single software product or be packaged into multiple software products.

In concluding the detailed description, those skilled in the art will appreciate that many variations and modifications can be made to the preferred embodiments without substantially departing from the principles of the present disclosure. Therefore, the disclosed preferred embodiments of the disclosure are used in a generic and descriptive sense only and not for purposes of limitation. The scope of protection of this disclosure should be interpreted in accordance with the claims below, and all technical ideas within the equivalent scope should be interpreted as being included in the scope of rights of this disclosure.

What is claimed is:

1. A method for generating an infectious disease prediction keyword, the method being performed by a computing apparatus, comprising:
obtaining a document including a target infectious disease as a corpus for each of a plurality of time sections;
converting a plurality of words included in the obtained corpus into embedding vectors;
calculating similarities between the respective converted embedding vectors and an embedding vector indicating the target infectious disease;
extracting an embedding vector of which the calculated similarity is higher than a predetermined first threshold value, for each time section;
obtaining first time series data indicating a search volume, over time, of a word corresponding to the embedding vector extracted for each time section;
calculating a correlation coefficient between the obtained first time series data and second time series data indicating the number of confirmed cases of the target infectious disease over time; and
generating a word corresponding to the first time series data of which the calculated correlation coefficient is higher than a predetermined second threshold value as a keyword for each time section.

2. The method of claim 1, wherein the converting of the plurality of words included in the obtained corpus into the embedding vectors and the calculating of the similarities are performed by a Word2Vec algorithm.

3. The method of claim 1, wherein the calculating of the correlation coefficient includes:
interpolating missing values of the first time series data and the second time series data;
normalizing the first time series data and the second time series data;
calculating correlation coefficients between the first time series data and the second time series data for each of a plurality of sliding windows; and
determining a maximum value of the calculated correlation coefficients as the correlation coefficient between the first time series data and the second time series data.

4. The method of claim 3, wherein the normalizing of the first time series data and the second time series data is performed by a min-max algorithm.

5. The method of claim 1, wherein the generating of the word corresponding to the first time series data of which the calculated correlation coefficient is higher than the predetermined second threshold value as the keyword for each time section includes:
calculating a p-value of the calculated correlation coefficient when the calculated correlation coefficient is higher than the second threshold value; and
generating a word corresponding to the first time series data of which the calculated p-value is lower than a predetermined third threshold value as the keyword for each time section.

6. The method of claim 5, wherein the generating of the word corresponding to the first time series data of which the calculated correlation coefficient is higher than the predetermined second threshold value as the keyword for each time section further includes determining a point in time when the correlation coefficient calculated within each time section is highest for the generated keyword.

7. The method of claim 1, further comprising training an infectious disease prediction model using the generated keyword.

8. The method of claim 7, wherein the infectious disease prediction model is implemented as a regression model, and
the training of the infectious disease prediction model includes regularizing a regularizer of the infectious disease prediction model.

9. The method of claim 7, further comprising displaying the number of confirmed cases of the target infectious disease on a user terminal, the number of confirmed cases of the target infectious disease being predicted by inputting a keyword generated for a period input by a user.

10. A computing apparatus comprising:
a processor; and
a memory storing instructions,
wherein when the instructions are executed by the processor, the instructions cause the processor to perform
obtaining a document including a target infectious disease as a corpus for each of a plurality of time sections;
converting a plurality of words included in the obtained corpus into embedding vectors;
calculating similarities between the respective converted embedding vectors and an embedding vector indicating the target infectious disease;
extracting an embedding vector of which the calculated similarity is higher than a predetermined first threshold value, for each time section;
obtaining first time series data indicating a search volume, over time, of a word corresponding to the embedding vector extracted for each time section;
calculating a correlation coefficient between the obtained first time series data and second time series data indicating the number of confirmed cases of the target infectious disease over time; and
generating a word corresponding to the first time series data of which the calculated correlation coefficient is higher than a predetermined second threshold value as a keyword for each time section.

11. The computing apparatus of claim 10, wherein calculating correlation coefficient includes:
interpolating missing values of the first time series data and the second time series data;
normalizing the first time series data and the second time series data;
calculating correlation coefficients between the first time series data and the second time series data for each of a plurality of sliding windows; and
determining a maximum value of the calculated correlation coefficients as the correlation coefficient between the first time series data and the second time series data.

12. The computing apparatus of claim 10, wherein generating the word corresponding to the first time series data of which the calculated correlation coefficient is higher than the predetermined second threshold value as the keyword for each time section includes:
calculating a p-value of the calculated correlation coefficient when the calculated correlation coefficient is higher than the second threshold value; and
generating a word corresponding to the first time series data of which the calculated p-value is lower than a predetermined third threshold value as the keyword for each time section.

13. The computing apparatus of claim 10, wherein when the instructions are executed by the processor, the instructions cause the processor to further perform:
training an infectious disease prediction model using the generated keyword; and
displaying the number of confirmed cases of the target infectious disease on a user terminal, the number of confirmed cases of the target infectious disease being predicted by inputting a keyword generated for a period input by a user to the infectious disease prediction model.

14. The computing apparatus of claim 13, wherein displaying the number of confirmed cases of the target infectious disease on the user terminal includes an operation of displaying the number of confirmed cases of the target infectious disease for a region input by the user on the user terminal.

15. The computing apparatus of claim 14, wherein displaying the number of confirmed cases of the target infectious disease on the user terminal further includes an operation of displaying information related to the number of confirmed cases of the target infectious disease for a keyword of interest input by the user on the user terminal.
